(19) United States
(12) Patent Application Publication
(10) Pub. No.: US 2017/0087270 A1
(43) Pub. Date: Mar. 30, 2017

(54) IMPROVED PRESSURE-SENSITIVE ADHESIVES USED FOR MEDICAL APPLICATIONS

(71) Applicant: Avery Dennison Corporation, Glendale, CA (US)

(72) Inventor: Anne Marie WIBAUX, Fontainebleau (FR)

(21) Appl. No.: 15/311,891

(22) PCT Filed: Jun. 15, 2015
(86) PCT No.: PCT/US2015/035787

§ 371 Date: Nov. 17, 2016

Related U.S. Application Data
(60) Provisional application No. 62/011,654, filed on Jun. 13, 2014.

(51) Int. Cl. A61L 15/58; A61F 13/02; C09J 7/02; A61L 26/00; C09J 133/08

(52) U.S. Cl. CPC A61L 15/585; A61L 26/008; A61L 26/0061; A61L 26/0052; C09J 133/08; C09J 7/0217; A61F 13/0213; A61F 13/0209; A61F 13/0253; A61F 13/0256; A61L 2420/06; A61L 2300/802; C09J 2401/00; C09J 2433/00

(57) ABSTRACT

Adhesive compositions are described which include one or more gelling agents in combination with one or more non-gelling disintegrants. The adhesive compositions exhibit relatively high fluid handling capacities and static absorption properties, thus enabling their use in a wide range of medical applications. Various adhesive articles such as dressings and related methods of use are also described which utilize the adhesive compositions. In preferred embodiments the adhesive component is an acrylic adhesive, the gelling agent is selected from the group consisting of (i) carboxymethyl cellulose, (ii) superabsorbent polymer, and (iii) combinations of (i) and (ii), and the non-gelling disintegrant is selected from the group consisting of (i) fully pregelatinized potato starch, (ii) microcrystalline celluloseand (iii) combinations of any of (i)-(ii).

### CROSS-REFERENCE TO RELATED APPLICATION

The present application claims the benefit of U.S. Provisional Application No. 62/011,654, filed Jun. 13, 2014, which is incorporated herein by reference in its entirety.

### FIELD

The present subject matter relates to pressure sensitive adhesives, articles using such adhesives, and related methods.

### BACKGROUND

Pressure sensitive adhesives used for medical applications such as in surgical dressings typically include hydrocolloids which form gels in the presence of water. The formation of a hydrophilic gel facilitates autolytic debridement and subsequent healing of wounds.

Gelling agents have been incorporated in medical adhesives to increase static absorption by the adhesive of water and fluids such as wound exudate. However, most gelling agents are relatively expensive and negatively impact the adhesive properties of the resulting composition. Also, hydrocolloid particles have been dispersed within an adhesive matrix. However, because adhesive matrixes are typically hydrophobic, transfer of moisture though the adhesive occurs when the hydrocolloid particles form bonds with each other. This is a limiting factor to the absorption and can require relatively high loadings of such particles. Accordingly, a need exists for a new strategy in which the static absorption and overall fluid handling capacity of an adhesive could be increased, without undesirable reductions in adhesive properties or requiring high loadings of hydrocolloid particles.

### SUMMARY

The present subject matter relates to a pressure sensitive adhesive which comprises at least one gelling agent and at least one non-gelling disintegrant. The incorporation of the non gelling disintegrant enhances transfer of moisture through the adhesive and enhances bridge formation between the gelling agent thereby leading to enhanced static absorption and fluid handling capacity.

In one aspect, the present subject matter provides an adhesive composition comprising at least one adhesive component, from 5% to 40% of at least one gelling agent, and from 5% to 40% of at least one non-gelling disintegrant.

In another aspect, the present subject matter provides an adhesive article comprising a thin film substrate defining a first face and a second face. The article also comprises an adhesive composition disposed on at least one of the first and second faces, the adhesive composition including (i) at least one adhesive component, (ii) from 5% to 40% of at least one gelling agent, and (iii) from 5% to 40% of at least one non-gelling disintegrant.

In yet another aspect, the present subject matter provides a method of promoting wound healing comprising providing an adhesive article including a thin film substrate and a layer of an adhesive composition disposed on the substrate. The adhesive composition includes (i) at least one adhesive component, (ii) from 5% to 40% of at least one gelling agent, and (iii) from 5% to 40% of at least one non-gelling disintegrant. The method also comprises applying the adhesive article on a wound such that the layer of the adhesive composition is directed toward and overlying the wound.

As will be realized, the subject matter described herein is capable of other and different embodiments and its several details are capable of modifications in various respects, all without departing from the claimed subject matter. Accordingly, the description is to be regarded as illustrative and not restrictive.

### DETAILED DESCRIPTION OF THE EMBODIMENTS

The present subject matter relates to pressure sensitive adhesive compositions. The adhesive compositions comprise (i) an adhesive component or matrix, (ii) one or more gelling agents, and (iii) one or more non-gelling disintegrants. The compositions exhibit relatively high static absorption (SA), moisture vapor transmission rate (MVTR), and fluid handling capacity (FHC) characteristics. In many embodiments and as described herein, static absorption and MVTR can be increased or enhanced by the addition of microcrystalline cellulose or other agents. The compositions also exhibit excellent compatibility with skin, thereby enabling their use in the medical field for adhering articles to patients.

The present subject matter also relates to adhesive articles, and particularly medical articles using the noted adhesive compositions. The adhesive articles comprise one or more substrates or layers, and at least one layer or region of the adhesive composition described herein.

The present subject matter additionally relates to methods of using the adhesive compositions described herein. The methods typically involve promotion of wound healing.

### Compositions

The compositions of the present subject matter will find wide application as fluid management adhesive compositions used in medical applications such as in controlling wound exudate. However, the present subject matter includes other applications and is not limited to the medical field. The compositions comprise one or more adhesive components, and a combination of one or more gelling agents and one or more non-gelling disintegrants. The use of a combination of gelling agent(s) with non-gelling disintegrants and in the proportions and the manner as described herein has been discovered to provide an array of beneficial properties in the resulting adhesive composition. In many embodiments, the adhesive compositions are highly breathable. The term “highly breathable” as used herein refers to the adhesive exhibiting a fluid handling capacity (FHC) of at least 2000 g/m²/24 hours or a MVTR of at least 800 g/m2/24 h.

### Adhesive Components

A wide array of adhesives can be used. In many embodiments, a solvent based adhesive is utilized. Non-limiting examples of such adhesives include acrylic adhesives, rubber adhesives, silicone adhesives, polyurethane adhesives, hybrid adhesives, and variants and combinations thereof. In certain embodiments, the adhesive is a pressure sensitive solvent based adhesive. Particularly, the adhesive is a pressure sensitive solvent based acrylic adhesive.

A description of useful pressure sensitive adhesives may be found in Encyclopedia of Polymer Science and Engineering, Vol. 13. Wiley-Interscience Publishers (New York, 1988). Additional description of useful pressure sensitive adhesives may be found in Encyclopedia of Polymer Science and Technology, Vol. 1, Interscience Publishers (New York, 1964).

The solvent-based acrylic adhesive may be any pressure sensitive adhesive that is capable of adhering to mammalian skin and that is free of ingredients known to cause undue irritation or toxicity to mammals. Useful acrylate copolymers may or may not be self-crosslinking and are formed from at least two monomers chosen from: (1) hydroxyalkyl esters of acrylic or methacrylic acid in which the alkyl group comprises 2 to 4 carbon atoms, such as 2-hydroxyethyl acrylate, 2-hydroxyethyl methacrylate, 2-hydroxypropyl acrylate and 2-hydroxypropyl methacrylate; (2) alkyl esters of acrylic or methacrylic acid in which the alkyl group of the ester comprises 4 to 18 carbon atoms, such as n-butyl acrylate or methacrylate, isopropyl acrylate or methacrylate, n-hexyl methacrylate and 2-ethylhexyl acrylate; (3) α,β-unsaturated monocarboxylic or dicarboxylic acids, their anhydrides and their alkyl or alkenyl esters in which the alkyl group contains from 1 to 3 carbon atoms and the alkenyl group contains from 2 to 5 carbon atoms, such as acrylic acid, itaconic acid, maleic acid, maleic anhydride, alkyl methacrylate and the diethyl esters of fumaric or maleic acid; (4) vinyl monomers, such as vinyl acetate, acrylonitrile, vinyl propionate, vinylpyrrolidone and styrene; (5) monomers containing a functional group selected from amido, amino and epoxy groups, for example, acrylamide, N-butylacrylamide, alkylarninoalkyl and aminoalky derivatives of acrylic or methacrylic acid, such as amino-ethyl acrylate, aminoethyl methacrylate and 2-(dimethylarnino) ethyl methacrylate, glycidyl methacrylate and glycidyl acrylate; (6) alkoxyalkyl esters of acrylic or methacrylic acid, for example methoxyethyl acrylates or methacrylates, butoxyethyl acrylates or methacrylates, methoxypropylene glycol acrylates or methacrylates and methoxypolyethylene glycol acrylates or rnethacrylates; and (7) hexamethylene glycol dimethacrylate. As these copolymers can be self-crosslinking, they may also contain a crosslinking agent selected from those generally used by those skilled in the art, for example, organic peroxides, polyisocyanates, chelates or metals such as titanium or aluminum, or metal acetylacetonates, such as those of zinc, magnesium and aluminum.

These adhesive acrylate copolymers may take the form of solutions in a solvent system consisting of a single organic solvent or a mixture of several solvents, which contain about 25% to about 55% by weight copolymers. Examples of suitable solvents include aromatic solvents such as toluene, xylene, etc. Suitable aliphatic solvents include esters such as ethyl acetate, propyl acetate, isopropyl acetate, butyl acetate, etc.; ketones such as methyl ethyl ketone, acetone, etc.; aliphatic hydrocarbons such as heptanes, hexane, pentane, etc. There can be included in the adhesive composition additive materials that do not affect the basic properties of the adhesive. Fillers, tackifiers, antioxidants, stabilizers, and the like may be added to the formulate adhesive. Further, pharmaceutically active components, such as for example, antimicrobials, anti-inflammatory agents, analgesic agents, anesthetics, or other pharmaceutically acceptable compounds, which do not affect the basic properties of the adhesive can be included in the adhesive layer in a pharmaceutically effective amount.

An example of a useful commercially available adhesive is DUROTACK 129A available from Henkel Corporation, which is a self-curing acrylic pressure sensitive adhesive containing 51% by weight solids in a solvent blend of heptane/isopropanol/ethyl acetate/toluene. Another example of a useful commercially available adhesive is DUROTAK 380-2819 available from Henkel, which is a self-crosslinking solution acrylic pressure sensitive adhesive containing 40% by weight solids in a solvent blend of ethyl acetate/isopropanol/heptanes/toluene/pentanedione.

Additional examples of adhesives and aspects thereof which may be suitable for use in the present subject matter, include those described in U.S. Pat. No. 7,078,582.

Although pressure sensitive adhesives are preferred, it will be understood that the present subject matter is not limited to such.

One or more rubber-based adhesives may be utilized. Non-limiting examples of preferred rubber-based adhesives include one or more styrene-isoprene-styrene polymers, styrene-olefin-styrene polymers including styrene-ethylene/propylene-styrene polymers, polyisobutylene, styrene-butadiene-styrene polymers, polyisoprene, polybutadiene, natural rubber, silicone rubber, acrylonitrile rubber, nitrile rubber, polyurethane rubber, polyisobutylene rubber, butyl rubber, halobutyl rubber including bromobutyl rubber, butadiene-acrylonitrile rubber, polychloroprene and styrene-butadiene rubber. Blends or mixtures of elastomers can be employed.

Various silicone adhesives and/or silicone gel adhesives can be used. Examples of silicone and/or silicone gel adhesives include but are not limited to those commercially available from Dow Corning Corp., Medical Products and those available from General Electric. Examples of silicone adhesives available from Dow Corning include those sold under the trade names BIO-PSA X7-3027, BIO-PSA X7-4919, BIO-PSA X7-2685, BIO-PSA X7-3122 and BIO-PSA X7-4502. Additional examples of silicone pressure sensitive adhesives useful in the present subject matter are described in U.S. Pat. Nos. 4,591,622, 4,584,355, 4,585,836 and 4,,655,767.

### Gelling Agents

Generally, the gelling agents selected for incorporation in the adhesive compositions include any substance which upon contact with moisture forms a gel. Typical gelling agents used in adhesive include materials such as carboxymethyl cellulose, hydroxyethyl cellulose, methyl cellulose, crosscarmellose sodium, starch glycolate and the like. In particular embodiments of the present subject matter, the gelling agents can be selected from one or more of (i) carboxymethyl cellulose, and (ii) crosscarmellose sodium.

An example of carboxymethyl cellulose is A800 carboxymethyl cellulose which is commercially available.

Croscarmellose sodium is an internally crosslinked sodium carboxymethyl cellulose. Specifically, croscarmellose sodium is the sodium salt of a crosslinked, partly O-(carboxymethylated) cellulose. An example of crosscarmellose sodium is PRIMELLOSE which is commercially available from DMV-Fonterra.

The present subject matter is not limited to the use of cellulose-derived materials for gelling agents. Instead, in certain embodiments, the compositions may comprise other gelling agents instead of, or in combination with, cellulose-derived materials. For example, suitable gelling agents include hydrocolloids such as gelatin, pectin, guar gum, locust bean gum, tragacanth gum, gum karaya, gum arabic, alginic acid and its sodium and/or calcium salts. Other synthetic hydrocolloids such as polyvinyl alcohol, polyvinyl acetate, polyvinyl pyrollidone, polyacrylic acid, polyhydroxyalkyl acrylates, polyacrylamides, high molecular weight polyethylene glycols and polypropylene glycols may be useful. Other hydrocolloids include crosslinked dextran and starch-acrylonitrile graft copolymer. Sodium starch glycolate is the sodium salt of a carboxymethyl ether of starch. The molecular weight of sodium starch glycolate is typically within a range of from 500,000 to 11,000,000.

In certain embodiments of the present subject matter, the gelling agent may include one or more super absorbent polymers (SAP).

The super absorbent polymers (SAP) useful in the adhesive composition comprise a water-swellable, hydrogel-forming absorbent polymer capable of absorbing large quantities of liquids such as water, body fluids (e.g., urine, blood), and the like. Additionally, the SAP is capable of retaining such absorbed fluids under moderate pressures. Typically the SAP absorbs many times its own weight in water, particularly at least 50 times, more particularly at least 100 times, and most particularly at least 150 times its weight in water. Additionally, the SAP exhibits good saline fluid absorption under load and high saline fluid absorption capacity. Typically the SAP absorbs at least 10 times, particularly at least 30 times, and more particularly at least 50 times its weight in saline fluid. Even though the SAP is capable of absorbing many times its own weight in water and/or saline, it does not dissolve in these fluids.

The ability of the SAP to absorb water and/or saline fluid is related to the degree of cross-linking present in the SAP. Increasing the degree of crosslinking increases the SAP's total fluid holding capacity under load. The degree of crosslinking is typically optimized to obtain a composition in which the rate and amount of absorbency are optimized. Particular SAPs are at least 10%, more particularly from about 10% to about 50%, and most particularly from about 20% to 40% crosslinked. Examples of suitable SAPs include crosslinked and polymerized α,β-beta ethylenically unsaturated mono- and dicarboxylic acids and acid anhydride monomers including, e.g., acrylic acid, methacrylic acid, crotonic add, maleic acid/anhydride, itaconic acid, fumaric acid, and combinations thereof.

Superabsorbent polymers useful in the present subject matter include, e.g., crosslinked acrylate polymers, crosslinked products of vinyl alcohol-acrylate copolymers, crosslinked products of polyvinyl alcohols grafted with maleic anhydride, cross-linked products of acrylate-methacrylate copolymers, crosslinked saponification products of methyl acrylate-vinyl acetate copolymers, crosslinked products of starch acrylate graft copolymers, crosslinked saponification products of starch acrylonitrile graft copolymers, crosslinked products of carboxymethyl cellulose polymers and crosslinked products of isobutylene-maleic anhydride copolymers, and combinations thereof.

The superabsorbent particles preferably are spherical and have an average particle size of from about 1 micrometer (μm) to about 400 (μm). In many embodiments the particles have an average particle size of from about 20 μm to about 200 μm, and particularly from 20 μm to 150 μm. In one embodiment, the particle size of the particles is less than 150 μm, or less than 100 μm. Useful commercially available superabsorbent particles include, e.g., sodium polyacrylate superabsorbent particles available under the AQUA KEEP series of trade designations including, e.g., particles having an average particle size of from about 20 μm to about 30 available under the trade designation AQUA KEEP 1 OSH-NF, particles having an average particle size of from 200 μm to 300 μm available under the trade designation AQUA KEEP 10SH-P, particles having an average particle size of from 320 μm to 370 μm available under the trade designation AQUA KEEP SA60S, particles having an average particle size of from 350 μm to 390 μm available under the trade designations AQUA KEEP SA60SX, SA55SX π and SA 60SL II, and particles having an average particle size of from 250 λm to 350 μm available under the trade designation AQUA KEEP SA6ON TYPE II from Sumitomo Seika Chemicals Col, Ltd. (Japan). Also available superabsorbent materials are Luquasorb 1010 and Luquasorb 1030 from BASF, Ludwigshafen, Germany.

In certain embodiments, compositions of the present subject matter include one or more gelling agents at a total weight proportion of from 5% to 40%, and in particular embodiments from 10% to 35%.

### Non-Gelling Disintegrants

The compositions of the present subject matter also include one or more non-gelling disintegrants. A disintegrant is an excipient used which when added to tablet induce breakup of tablet when it comes in contact with aqueous fluid to facilitate the drug release. The term “non-gelling disintegrant” as used herein refers to a disintegrant which does not form a gel during and upon contact with fluid. Specifically, the non-gelling disintegrants serve to promote absorption and passage of water through the adhesive compositions, increasing overall porosity of the composition, and/or increasing capillary action or wicking of the composition; while not forming a gel. The non-gelling disintegrants can be selected from one or more of (i) microcrystalline cellulose, (ii) potato starch, and (iii) modified starches. Combinations of (i)-(iii) can also be used.

A wide array of microcrystalline cellulose materials can be used. An example of such material is AVICEL PH105 which is commercially available from FMC Biopolymer.

A wide array of potato starches can be used. The potato starch is subjected to one or more operations to fully pregelatinize the potato starch. Typically, operations such as heating the starch molecules in the presence of water can be utilized to thereby allow hydrogen bonding sites in the molecule to engage more water. This irreversibly dissolves the starch granule. The term “pregelatinized starch” as used herein refers to starch that has been chemically and/or mechanically processed to rupture all or part of the starch granules in the presence of water and subsequently dried. For example, a fully pregelatinized starch can be prepared by controlled thermal pregelatinization of potato starch followed by spray drying. Typically fully pregelatinized starch is extremely soluble in cold water. An example of fully pregelatinized potato starch is PREJEL which is commercially available from DMV-Fonterra. An example of chemically crosslinked and carboxymethylated potato starch is PRIMOJEL which is commercially available from DMV-Fonterra.

In certain embodiments, the compositions of the present subject matter include one or more non-gelling disintegrants at a total weight proportion of from 5% to 40%, and in particular embodiments from 15% to 35%.

The non-gelling disintegrant(s) can be utilized at various particle sizes. In many embodiments of the present subject matter, the non-gelling disintegrant(s) is selected such that upon incorporation in the composition, the disintegrant(s) is effectively incorporated within the adhesive component. In many embodiments, particle sizes of less than about 200 microns have been found to be useful. For example, in certain embodiments the non-gelling disintegrants are in a particulate form and have an average particle size of from about 1 micron to about 150 microns, more particularly within a range of from 10 to 100 microns, and in certain embodiments within a range of from 20 to 80 microns. However, the present subject matter includes the use of particle sizes of the non-gelling disintegrant(s) having a particle size of 20 microns or greater.

Although the non-gelling disintegrant(s) has been described as one or more of (i) potato starch and particularly fully pregelatinized potato starch and (ii) microcrystalline cellulose it will be understood that the present subject matter includes the use of other suitable non-gelling disintegrants.

### Additional Additives

One or more additional additives can be incorporated into the adhesive compositions. Particularly, the additional additives include medicinal compounds. Such medicinal compounds include, but are not limited to, antirnicrobials, antibiotics, antifungal agents, antiviral agents, antithrornbogenic agents, anesthetics, anti-inflammatory agents, analgesics, anticancer agents, vasodilation substances, wound healing agents, angiogenic agents, angiostatic agents, immune boosting agents, growth factors, and other biological agents. Suitable antimicrobial agents include, but are not limited to, biguanide compounds; triclosan; penicillins; tetracyclines; aminoglycosides, such as gentamicin and Tobramycin™; polymyxins; rifampicins; bacitracins; erythromycins; vancomycins; neomycins; chloramphenicols; miconazole; quinolones, such as oxolinic acid, norfloxacin, nalidixic acid, pefloxacin, enoxacin, and ciprofloxacin; sulfonamides; nonoxynol 9; fusidic add; cephalosporins; and combinations of such compounds and similar compounds. The additional antimicrobial compounds provide for enhanced antimicrobial activity.

In one embodiment, the pressure sensitive adhesive contains one or more medicament(s) capable of being transdermally absorbed or delivered onto the skin surface. As used herein, the term “medicament” refers to a composition comprising at least one active ingredient, which may be added to the pressure sensitive adhesive layer together with a pharmaceutically acceptable vehicle suitable for cutaneous application. In certain embodiments, the medicament may further comprise one or more excipients including, but not limited to preservatives, antioxidants, moisturizers, emollients, buffering agents, solubilizing agents, penetration enhancers, skin protectants, and mixtures thereof.

### Adhesive Articles

The present subject matter also includes a wide array of adhesive articles and particularly adhesive medical articles using the noted compositions. The articles comprise one or more substrates or other layers or components, one or more layers or regions of the noted adhesive compositions, and typically, a liner or liner assembly covering the layer or region of the adhesive composition. The articles may optionally comprise one or more cover or backing layers or components.

### Substrate or Other Layer(s)

Substrate materials include but are not limited to elastomeric polyurethane, polyester, or polyether amide films. Desirable properties include high moisture vapor and oxygen permeability, resiliency, conformability and in certain embodiments, transparency. Disposable secondary backing films such as polypropylene (PP) or polyethylene (PE) can be used to provide additional ease of handling. Alternatively, breathable paper or textile backings may also be appropriate for use. Additional examples of such paper or textile backings are provided in “A Review on Designing the Waterproof Breathable Fabrics,” A. Mukhopadhyay; V. K. Midha, Journal of Industrial Textiles, Part 1-37, 225 (2008) & Part II—38, 17 (2008) and in U.S. Pat. No. 6,495,229 and related patents.

A wide array of materials can be used for one or more cover or backing layer(s) such as but not limited to, polyurethane, paper, polyethylene, polypropylene, and other polymeric film layers. Particular aspects for each of these materials are as follows.

Polyurethane—Melt-blown non-woven fibrous webs comprised of three-layer polymeric fibers having a center layer of blended polyethylene and KRATON, pressure sensitive adhesive, and outer layers of polyurethane; prepared as described for Backing Sample 16 in U.S. Pat. No. 6,107,219 to Joseph et al.; represents a suitable elastic, non-tearable backing or cover layer.

Paper—Hammermill Laserprint paper (0.11 mm thick, Product No. 00460-4, International Paper, Memphis, Tenn.); represents a typically non-stretchable, tearable backing.

Polyethylene—Melt-blown non-woven polyethylene (0.04 mm thick, Style No. TM07-27-98-02, Trans Web LLC, Vineland, N.J.); represents a typically stretchable, tearable backing.

Polypropylene—Melt-blown non-woven polypropylene (basis weight 20 g/m², Kimberly Clark, Irving, Tex.); reproyentya typically non-stretchable, tearable backing.

Film—Polymer film comprising 60% ethylene/vinyl acetate copolymer, 35% linear low density polyethylene, 5% stabilizers and other additives (PGI Product No. 6012, Polymer Group, c., Gainesville, Ga.); film had a basis weight of 1.15 oz/yd² (27 g/m²), was 5 mils (0.13 mm) thick, and had oval-shaped holes (approximately 0.2 mm width by 0.3 mm length in the greatest dimensions) with the length dimension of the oval holes oriented parallel to the machine direction of the film. The film had about 530 holes/cm² arranged in a pattern of staggered lines. One side of the film was “smooth” (microetchediembossed for smoothness) and the other side was “rough” (side that had material pushed out from forming the holes).

Additional details of various materials suitable for cover layers are described in U.S. Pat. No. 7,078,582. A preferred substrate would be water proof, have a breathability of at least 800 g/m2/24 hours and would have a thickness from 0.20 to 3 mil.

### Adhesive Layer or Region

The adhesive articles comprise one or more layers or regions of the noted adhesive compositions. The adhesive layers or regions are applied or otherwise deposited on the substrate by known application techniques. Typically, the adhesive is applied in a layer form having a thickness of from 0.01 mm to about 5 mm or thicker, and in particular embodiments within a thickness range of from 0.05 mm to 2 mm. The surface coverage of the adhesive can vary from 5% (in which case this would be a pattern coating) to 100% (fully coated substrate).

### Liner

Suitable materials for liners include those made with kraft papers, polyester, polypropylene (PP), polyethylene (PE) or other composite constructions. The release coatings are preferentially coated with low energy materials like silicone, fluorochernicals, etc. that afford beneficial performance. Examples of representative silicone materials are provided in Chapter 18, Handbook of Pressure Sensitive Adhesives, Van Nostrand Reinbhold, 1982, page 384. Examples of suitable fluorochernicals are described in U.S. Pat. No. 4,472,480.

The present subject matter includes various combinations of these materials. The particular selection and application will be appreciated by those skilled in the art in designing functional laminates using optimum facestock, adhesive and liner combinations.

The present subject matter articles as described herein can be incorporated in or used in conjunction with a wide array of medical products. Representative examples of such products include, but are not limited to bandages, dressings, gauze, tape and related products, wound closure products such as patches, covers and the like, closure strips foam padding, surgical tapes, and pads. Numerous applications are contemplated in which the present subject matter articles are incorporated in and/or used in conjunction with vacuum assist closure (VAC) products and therapies.

### Methods

The present subject matter also provides various methods using the noted compositions. The methods are directed to promoting wound healing, which may also include absorbing fluids such as wound exudate, and fluid management applications. Generally, the methods comprise one or more operations of providing an adhesive article such as described herein which includes at least one layer or region of the noted adhesive composition. Typically, the adhesive article is in the form of a dressing. The methods also comprise one or more operations of applying the adhesive article onto a biological surface such as a wound area exposed along a patient's skin or other body region. The article is applied such that the adhesive layer or region is directed towards, and in many applications, contacts the wound area. As a result of the unique fluid handling characteristics of the noted compositions, the methods achieve one or more of promotion of wound healing, absorbing fluids such as wound exudate, and managing or controlling fluids in and around a wound.

### Examples

A series of trials were conducted to evaluate the compositions of the present subject matter. The compositions included an adhesive component, a gelling agent, and a non-gelling disintegrant. The adhesive was DUROTAK 129A commercially available from Henkel. The gelling agent was selected as A800 carboxymethyl cellulose commercially available from various suppliers. The non-gelling disintegrant was selected from microcrystalline cellulose available under the designation AVICEL PH105 from FMC Biopolymer.

Referring to Table 1 set forth below, samples 1-9 were prepared using the noted components in the designated weight percentages:

TABLE 1
Summary of Test Results of Samples 1-9

| | | | | | Additive | Additive | Coat | MVTR | SA | FHC |
|---|---|---|---|---|---|---|---|---|---|---|
| | | Additive | Additive | Adhesive | No. 1 | No. 2 | Weight | (g/m²/ | (g/m²/ | (g/m²/ |
| Samples | Adhesive | No. 1 | No. 2 | (% w/w) | (% w/w) | (% w/w) | (gsm) | 24 hours) | 24 hours) | 24 hours) |
| 1 | Durotak | A800 | N/A | 65 | 35 | 0 | 100 | 1250 | 690 | 1940 |
| | 129A | | | | | | | | | |
| 2 | Durotak | A800 | N/A | 65 | 35 | 0 | 100 | 1140 | 600 | 1740 |
| | 129A | | | | | | | | | |
| 3 | Durotak | A800 | N/A | 65 | 35 | 0 | 100 | 1090 | 610 | 1700 |
| | 129A | | | | | | | | | |
| 4 | Durotak | A800 | Avicel | 50 | 35 | 15 | 101 | 1340 | 1516 | 2856 |
| | 129A | | PH105 | | | | | | | |
| 5 | Durotak | A800 | Avicel | 50 | 35 | 15 | 101 | 1340 | 1515 | 2855 |
| | 129A | | PH105 | | | | | | | |
| 6 | Durotak | A800 | Avicel | 50 | 35 | 15 | 101 | 1341 | 1517 | 2858 |
| | 129A | | PH105 | | | | | | | |
| 7 | Durotak | Avicel | N/A | 60 | 40 | 0 | 100 | 1200 | 200 | 1400 |
| | 129A | PH105 | | | | | | | | |
| 8 | Durotak | Avicel | N/A | 60 | 40 | 0 | 100 | 1300 | 200 | 1500 |
| | 129A | PH105 | | | | | | | | |
| 9 | Durotak | Avicel | N/A | 60 | 40 | 0 | 100 | 1400 | 200 | 1600 |
| | 129A | PH105 | | | | | | | | |

For samples 1-3 the evaluated compositions included the A800 component as the gelling agent without any other components. In samples 7-9, the evaluated composition included the AVICEL PH105 as the disintegrant component without any other components. Samples 4-6 evaluated a combination of gelling agent and non gelling disintegrant. Samples 4-6 correspond to an embodiment of the present subject matter.

The following test methods were used to evaluate the samples noted in Table 1:

TABLE 2
Test Methods

| Test | Standard |
|---|---|
| 180° Peel | — |
| Moisture Vapor Transmission | T06/022, EN13726, ASTM E 96-80 |
| Rate (MVTR) | |
| Static Absorption (SA) | T06/022, EN13726, ASTM E 96-80 |
| Fluid Handling Capacity (FHC) | T06/022, EN13726, ASTM E 96-80 |

### 180 Degree Peel

Samples of the adhesive either directly coated on PET film or laminated to PET film from a release liner were cut into about 2.54 cm by about 20 cm test strips. Adhesive samples were rolled down on a test panel of stainless steel, HDPE or cardboard with a 2 kg rubber clad steel roller moving back and forth at a rate of about 30 cm/min. After a dwell time of 24 hours, the test strips were peeled away from the test panel in an Instron Tensile Tester at 180 degree to the test panel, i.e., folded back on itself and parallel to the surface of the panel, at a rate of about 30 cm/min. The force to remove the adhesive strip from the test panel was measured in Newtons per meter (N/m). Tests were performed in triplicate and the average value was reported.

### Moisture Vapor Transmission Rate, Static Absorption, and Fluid Handling Capacity

Methods for measuring moisture vapor transmission rate (MVTR), static absorption (SA), and fluid handling capacity (FHC) are set forth in T06/022 (which corresponds to EN13726 and ASTM E 96-80), and are generally as follows.

Fluid Handling Capacity is a measure of the combined ability of the composite to take up moisture and to evaporate it to the environment. Moisture vapor transmission rate (MVTR) measures the rate at which moisture permeates through a wound dressing measured in grams/meter/day. The static absorption measures the amount of fluid uptake into a known surface. This test is performed by laminating a sample cut to the size of a Paddington cup to the cup on the side having the rubber ring. The circular sealing ring is placed on the sample of the cup and the screws are secured. The cup is weighed (W1). The cup is then turned upside down and filled with 20 ml of a NaCl solution (0.9% wt in deionized water). The metal sealing place is secured to the top side of the cup. The filled cup is weighed (W2). The cup is placed sample side down into an oven at 37° C. For 24 hours. After 24 hours, the cup is removed from the oven and allowed to cool to room temperature for 30 minutes. The cup is then weighed (W3). The metal sealing plate is removed and the cup is emptied. The cup is allowed to stand for 15 minutes on a tissue to remove the NaCl solution, and then weighed (W4). The test conditions are 23° C. (±2°) and 50% (±2%) relative humidity. The Moisture Vapor Transmission Rate (MVTR) equals (W2−W3)×1000. The Static Absorption equals (W4−W1)×1000. The Fluid Handling Capacity (FHC) in g/m²/24 hours is determined as follows:

FHC=(W2−W3)+(W4−W1)

Referring to Table 1, it can be seen that adhesive compositions according to the present subject matter, in examples 4-6, with the gelling agent and non-gelling disintegrant, exhibit relatively high moisture vapor transmission rates (MVTRs), such as for example greater than 1320 g/m²/24 hours; relatively high static absorption properties, such as for example greater than 1450 g/m²/24 hours; and relatively high fluid handling capacities, such as for example greater than 2000 g/m²/24 hours, in certain embodiments greater than 2200 g/m²/24 hours, in certain embodiments greater than 2400 g/m²/24 hours, in certain embodiments greater than 2600 g/m²/24 hours, and in particular embodiments greater than 2800 g/m²/24 hours.

Use of the non-gelling disintegrant(s) in combination with the gelling agent(s) as described herein can in certain embodiments, serve to increase static absorption and fluid handling capacity of the adhesive composition. Thus, a formulator can save costs by using less gelling agent and still achieve the same or higher level of static absorption, MVTR and fluid handling capacity by incorporating the non-gelling disintegrant(s). Additionally, in using the non-gelling disintegrant(s), the amount of gelling agent can be reduced in certain applications, which thereby increases the initial tack of the pressure sensitive adhesive.

Compositions according to the present subject matter were also evaluated with regard to 180 Degree Peel characteristics. The compositions exhibited relatively low 180° peel values, such as for example less than 0.196 N/m. This indicates their potential usefulness as skin adhesives in medical articles that can be removed from skin without damaging the skin.

Many other benefits will no doubt become apparent from future application and development of this technology.

All patents, applications, standards, and articles noted herein are hereby incorporated by reference in their entirety.

The present subject matter includes all operable combinations of features and aspects described herein. Thus, for example if one feature is described in association with an embodiment and another feature is described in association with another embodiment, it will be understood that the present subject matter includes embodiments having a combination of these features.

As described hereinabove, the present subject matter solves many problems associated with previous strategies, systems and/or devices. However, it will be appreciated that various changes in the details, materials and arrangements of components, which have been herein described and illustrated in order to explain the nature of the present subject matter, may be made by those skilled in the art without departing from the principle and scope of the claimed subject matter, as expressed in the appended claims.

1. An adhesive composition comprising:
at least one adhesive component;
from 5% to 40% of at least one gelling agent; and
from 5% to 40% of at least one non-gelling disintegrant.

2. The composition of claim 1 wherein the adhesive component is a pressure sensitive adhesive.

3. The composition of claim 1 wherein the adhesive component is a solvent based adhesive.

4. The composition of claim 1 wherein the adhesive component is an acrylic adhesive.

5. The composition of claim 1 wherein the gelling agent is selected from the group consisting of (i) carboxymethyl cellulose, (ii) superabsorbent polymer, and (iii) combinations of (i) and (ii).

6. The composition of claim 1 wherein the non-gelling disintegrant is selected from the group consisting of (i) fully pregelatinized potato starch, (ii) microcrystalline celluloseand (iii) combinations of any of (i)-(ii).

7. The composition of claim 1 further comprising:
at least one additional additive.

8. The composition of claim 1 wherein the composition exhibits a fluid handling capacity (FHC) of at least 2000 g/m²/24 hours.

9. The composition of claim 8 wherein the composition exhibits a fluid handling capacity of at least 2200 g/m²/24 hours.

10. The composition of claim 8 wherein the composition exhibits a fluid handling capacity of at least 2400 g/m²/24 hours.

11. The composition of claim 8 wherein the composition exhibits a fluid handling capacity of at least 2600 g/m²/24 hours.

12. The composition of claim 8 wherein the composition exhibits a fluid handling capacity of at least 2800 g/m²/24 hours.

13. The composition of claim 1 wherein the composition exhibits a moisture vapor transmission rate (MVTR) of at least 800 g/m²/24 hours; and a static absorption (SA) of at least 500 g/m²/24 hours.

14. The composition of claim 1 wherein the composition exhibits a moisture vapor transmission rate (MVTR) of at least 1320 g/m²/24 hours; and a static absorption (SA) of at least 1450 g/m²/24 hours.

15. The composition of claim 1 wherein the composition exhibits a 180° peel value of less than 0.196 N/m.

16. An adhesive article comprising:
a thin film substrate defining a first face and a second face; and
the adhesive composition of claim 1, wherein the adhesive composition is disposed on at least one of the first and second faces.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. The article of claim 16 wherein the adhesive composition is disposed on the substrate in a layer and the layer has a thickness within a range of from 0.05 mm to 2 mm.

31. A method of promoting wound healing comprising:
providing an adhesive article of claim 16 and;
applying the adhesive article on a wound such that the layer of the adhesive composition is directed toward and overlying the wound.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. The method of claim 31 wherein the adhesive composition is disposed on the substrate in a layer and the layer has a thickness within a range of from 0.05 mm to 2 mm.
